(19) United States
(12) Patent Application Publication
(10) Pub. No.: US 2006/0178590 A1
(43) Pub. Date: Aug. 10, 2006

(54) Method and apparatus for monitoring bariatric parameters and sleep disordered breathing

(76) Inventors: Malcolm Ronald Hebblewhite, New South Wales (AU); Kleran Gallahue, San Diego, CA (US); David Mulcahy, New South Wales (AU); Hary Soesanto Surjadi, New South Wales (AU); Benriah Goeldi, New South Wales (AU); Ron Richard, Poway, CA (US); Deirdre Stewart, San Diego, CA (US)

Correspondence Address: GOTTLIEB RACKMAN & REISMAN PC, 270 MADISON AVENUE, 8TH FLOOR, NEW YORK, NY, 100160601, US

(21) Appl. No.: 10/560,963

(22) PCT Filed: Jun. 18, 2004
(86) PCT No.: PCT/AU04/00796

§ 371 Date: Mar. 30, 2006

Related U.S. Application Data
(60) Provisional application No. 60/479,320, filed on Jun. 18, 2003.

(51) Int. Cl. A61B 5/08

(52) U.S. Cl. 600/529; 128/920

(57) ABSTRACT

A method and system is disclosed for monitoring SDB managment that compares titrated CPAP pressure against BMI and AHI/AI indices. The method comprises the steps of storing data on a computer, where the data is collected over a time period of interest. The data consists of BMI and one or more of AHI, AI, Usage and CPAP titration measurements. The method further comprises illustrating the stored data for a selected time period.

### FIELD OF THE INVENTION

This invention relates to ventilatory assistance for respiratory needs and more specifically to relationships between obstructive sleep apnea, body mass index, continuous positive airway pressure therapy and compliance measures.

### BACKGROUND OF THE INVENTION

Obstructive sleep apnea (OSA) is a common form of sleep disordered breathing (SDB). There is a clinically recognized relationship between a patient's body mass and the likelihood of the patient suffering from OSA. The clinically recognised measure of relative body mass is known as the body mass index (BMI). Specifically, the BMI is calculated as the weight of the patient, in pounds, divided by the square of the height of the patient, in inches, with the result being multiplied by the conversion factor of 703. In SI units, the BMI is calculated from the patient's weight in kilograms divided by the patient's height in metres squared. The greater the BMI, the more likely the patient is to suffer from OSA. Accordingly, an OSA sufferer may be advised to reduce his BMI as a step in the management of his OSA condition.

To reduce his BMI, a patient may be required to engage in routine and vigorous exercise and/or may be placed on a strict diet. Strict diet control can be achieved by the patient voluntarily limiting his intake of the amount of food, especially food associated with an increase in BMI such as fats. As an aid to controlling food intake, a patient may undergo bariatric surgery where his stomach capacity is reduced and/or bypassed so as to reduce the amount of food that may be digested in a given period.

Aside from altering the BMI, other techniques exist for treating OSA. One type of treatment is non-invasive positive pressure ventilation, or NIPPV. NIPPV delivers ventilatory support without the need for known invasive artificial airway procedures. A specific type of NIPPV is continuous positive airway pressure (CPAP). CPAP is known for being almost 100% effective in treating OSA. CPAP delivers air into a patient's airway through a specially designed device, such as a nasal mask. Air pressure in the mask, measured in cm of H₂0, forces the airway to remain open.

During CPAP treatment, applied pressure may be titrated (adjusted) to maintain the airway patency, or quality of the airway opening. The titration process may be manual or automatic. Various patents have focused on automatic titration systems, including U.S. Pat. Nos. 5,704,345, 6,363,933, 6,532,959, 6,484,719 and 6,532,957, the contents of all of which are hereby incorporated by reference.

Automatic titration systems have a capacity to collect, store and display data regarding a patient's breathing by recording the patient's apnea hypopnea index or apnea index (AHI or AI) where the hypopnea index is a count of partial closings of the breathing path while the apnea index is a count of the complete cessations of breathing.

### SUMMARY OF THE INVENTION

A method and system is disclosed for monitoring SDB management that compares titrated CPAP pressure against BMI and AHI/AI indices. The method comprises the steps of storing data on a computer, where the data is collected over a time period of interest. The data consists of BMI and one or more of AHI, AI, Compliance and CPAP titration measurements. The method further comprises illustrating the stored data for a selected time period.

### BRIEF DESCRIPTION OF THE DRAWINGS

The features and advantages of the present invention will become more readily apparent from the following detailed description of the invention in which:

FIG. 1 is a screenshot illustrating the data layout according to the invention; and

FIG. 2 is a flow chart displaying the process of the invention.

### DETAILED DESCRIPTION OF THE PREFERRED EMBODIMENTS

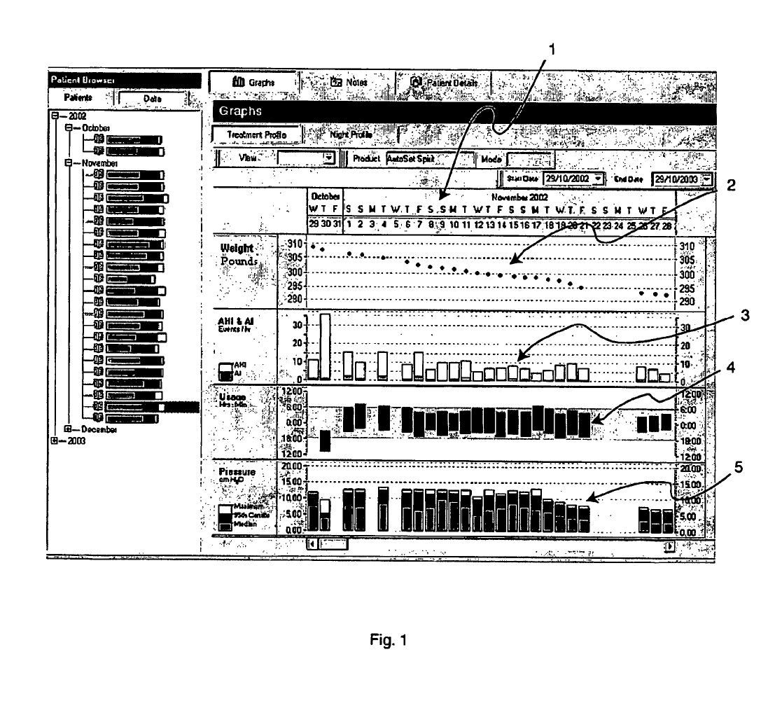

Turning to FIG. 1, a method is disclosed for assisting in SDB management. The method includes monitoring and graphing, for a time period of interest, BMI 2, AHI/AI 3, CPAP titration 5 and compliance 4. This embodiment has been implemented in the AUTOSET SPIRIT™ micro-processor controlled flow generator and AUTOSCAN™ software application by ResMed Limited. The AUTOSCAN™ is a PC application, which downloads data from the AUTOSET SPIRIT™ to be viewed by a clinician. Accordingly this invention can be implemented in a flow generator or a stand-alone computer that may communicate with a flow generator.

The screenshot of FIG. 1 includes a first horizontal pane 1 representing a selected time period. The time period represents, for example, a series of sessions identified as single days. The purpose of the pane 1 is to allow a clinician or patient (if patient access is allowed) to monitor information on a daily basis.

In the pane 1, the series of days represents one calendar month, from October 29 through November 28. Typically, the last day would be the current date. However, the current day could be prior to the last date on the display, with data for the future dates being statistically calculated by the system based on exhibited trends. Yet alternatively, the entire series of days displayed could represent archived data retrieved for purposes of review.

The screen-shot left-vertical pane of the screen provides an index of each day's flow generator usage and allows a period to be selected

A second horizontal pane 2 depicts the BMI of the patient. An individual BMI value is recorded for each day. Plotting the BMI allows the clinician or patient to determine if the BMI is following a suggested or required program. BMI values are and the system displays BMI, on the right side of pane 2, in the range of 44 to 47.

BMI data is manually input into the computer using typical input devices, such as a keyboard or other device requiring active user input. The BMI data may be entered daily, weekly, bi-weekly, monthly or at some other time interval. In a preferred embodiment it is entered every two weeks. Alternatively, the system is capable of automatically calculating a BMI value after receiving weight and height information. A sliding scale for both weight and BMI measures are respectively displayed on the left and right sides of the BMI pane.

Pain 2 could also include a trace that represents an ideal BMI progression or a band representing the desirable range between which the BMI should progress. Pain 2 could also include a further band or bands representing a further range existing beyond the desirable range and into which it would be undesirable for the BMI to progress. By the inclusion of these additional traces and bands pain 2 provides a visual indication of condition management targets and the patient's actual progress. Such information could be made accessible by the patient as the clinician determines. Suitable labels can be displayed alongside the data such as “Normal”, “Overweight”, “Obese”, etc. These may be displayed only to the clinician, or to both the patient and the clinician. Additional or alternative labels may be displayed to the patient.

A third horizontal pane 3 on the screen illustrates the AHI/AI values for each day (CPAP session). As shown by the legend on the left side of the pane, the AHI and AI values are respectively plotted in white and black. The purpose of displaying the AHI against the AI is to determine the severity of the patient's condition. In the illustration, the data indicates that the number of AHI occurrences is greater than the number of AI occurrences for each treatment day. As an example, on October 30, there were more than 30 hypopnea occurrences and fewer than 10 apnea occurrences.

The units displayed for the AHI/AI, on the right side of the pane 3, are in numbers of occurrences per session. The purpose of displaying both AHI and AI against BMI is to determine the correlation between a changing BMI and the AHI/AI. This helps determine if the patient is responding according to statistics that normally apply to the overall patient population.

The system may receive AHI/AI data manually as with the BMI. Alternatively, the system may receive AHI/AI data automatically, by known monitoring methods. By way of example the data may be acquired, stored, collated and processed and distributed by way of a patient compliance management system as described in U.S. Provisional Patent Application No. 60/500,866 filed 5 Sep. 2003 the contents of which is hereby incorporated by reference.

A fourth horizontal pane 4 on the graph illustrates the usage or compliance of the patient regarding each CPAP session. Individual indicators represent durations of time as well as the starting time (bottom of indicator) and ending time (top of indicator) of the usage during specific sessions. The purpose of graphing usage time is to determine the effect that duration of a CPAP session has on the AHI/AI measures. The time scale is indicated on the left side of the pane 4. A missing indicator illustrates an omitted session for the day. For example, there was no CPAP session on October 31.

The system is capable of receiving information as to the usage manually, as with the BMI. Alternatively, the system is capable of automatically receiving information of the usage of the CPAP device. For example, the CPAP device is capable of being monitored by the computer to determine if the device is turned on and off.

A fifth horizontal pane 5 illustrates the CPAP titration. Each day has an associated CPAP titration indicator used to illustrate the pressures required for a successful CPAP application. The titration is displayed in typical terms of cm of H₂O of pressure (left side of pane 5). In the illustration, the pressure ranges from zero to twenty cms of H₂O. For each session, CPAP titration is displayed in terms of maximum pressure, median pressure, and the level below which the pressure was 95% of the time (the ninety fifth percentile).

As an example of correlations between the variables, consider the data recorded on October 29 and October 30. On those days, the maximum CPAP differed slightly, while the ninety fifth percentile differed significantly. Over the same days, the AI readings were essentially identical while the AHI readings differed significantly. Further, the usage time differed significantly from the norm. By providing the information to the clinician, the system enables the clinician to determine how these variables relate to each other.

Through the graphed variables, the system enables the clinician to identify whether the patient's physiology follows generally known trends. For example, it would be generally expected that decreasing the BMI of the patient would allow for a decreased CPAP. In the illustration, this downward trend was followed from November 12 to November 28, representing almost half of the recorded treatment for the month. Accordingly, the actual trend for the patient confirmed the expected trend.

The system is capable of receiving titration data automatically or manually using known techniques. The system is capable of automatically determining AHI/AI indices by monitoring the output of microcontrollers, such as those used for controlling CPAP titration in U.S. Pat. No. 6,532,959, incorporated above.

It would be useful to store, in a standard personal computer, BMI data, CPAP titration data and, for example, AHI/AI data for a particular patient, and chart the stored data. The charted information would enable the determination of the accuracy of presumed physiological correlations. For example, such a system could affirm that as the patient's BMI decreases, the CPAP pressure also decreases. Alternatively, the system could identify other physiological relationships and trends that affect particular persons or groups of persons.

The invention may be used in the management of patients with type 2 diabetes. The mainstay of treatment for Type 2 diabetes is dietary and lifestyle changes. In patients with residual hyperglycaemia, oral medications, and less commonly, parenteral insulin may be required. The disease is chronic and regular monitoring is undertaken to determine the adequacy of glycemic control and the possible development of secondary complications such as retinopathy, nephropathy and peripheral neuropathy. While currently, there is no established role for CPAP in the treatment of type 2 diabetes, OSA frequently coexists with diabetes (obesity being a common predisposing factor), and in patients with both illnesses the sympathetic activation caused by OSA is thought to potentially worsen diabetic control. The current evidence shows that insulin resistance (a condition associated with obesity, characterized by overproduction of insulin and a precursor of diabetes) can be improved by treatment with CPAP in patients with coexisting OSA. Accordingly, the present invention may be readily implemented so as to allow for the monitoring and collating of data relevant to the treatment of patients with type 2 diabetes including the compliance with and effect of CPAP treatment.

Once physiological relationships and trends are ascertained, the comparison of treatment results with those of other OSA management procedures would enable the determination of the relative effectiveness of the procedures.

The display of information in the manner shown in each pane of FIG. 1 serves to allow for the convenient recognition of trends and the possible correlations between physiological phenomena. When the data is considered in the context of the patient's overall clinical management regime, it is possible to readily determine the effectiveness or otherwise of the BMI modifying treatment and OSA management.

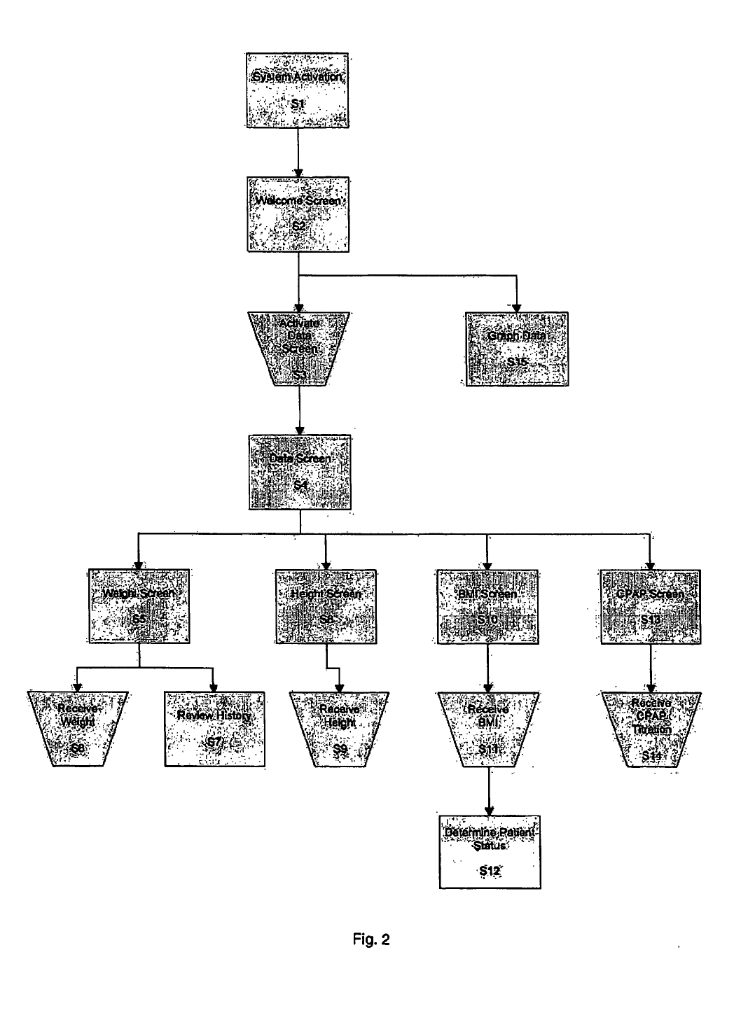

Turning to FIG. 2, an example of how the invention may be operated is now described. At step S1, the components that monitor BMI, AHI/AI, usage and CPAP titration are activated, and the system software is activated. At step S2, the system presents a welcome screen. Upon reaching the welcome screen, the system enables the user to use an input device, such as a mouse, and activate a data screen at step S3.

In response to the user input at step S3, the system activates the data screen at S4. Through the data screen, the system provides the user with a weight screen at step S5, where the system receives the weight for the day at step S6. The weight is supplied either automatically or manually, as discussed.

For manual operation, after the initial activation, the system provides the user with the previously recorded weight for the patient. The weight is capable of being modified in the system by increments of one pound in response to the system receiving input via the customary keyboard arrows. This feature allows for convenient data entry as it may be expected that while a patient's weight will vary over time, it will vary by no more than a few pounds between consecutive sessions. The weight data may be entered on a daily basis or on another period such a weekly or monthly as is considered clinically appropriate.

While in the weight screen, the system allows the user to review the weight for a selected period of time, such as 30 days, in step S7. The purpose of this review screen is to determine if the BMI for the patient is in accordance with a predefined schedule. If a weight was not entered on a day it will show “N/A” for Not Available.

The system allows the user to return to the data screen whereupon the user is capable of entering a height screen at step S8. The user could have also entered the height screen initially from the data screen following step S4. The height screen, through automated or manual input, allows the system to receive the height of the patient, in inches in step S9.

The system allows the user to return to the data screen whereupon the user is capable of entering a BMI screen at step S10. As with the height screen, the user could have also entered the BMI screen initially from the data screen following step S4. The BMI screen, through automated or manual input, allows the system to receive BMI data at step S11. Step S10 will often be unnecessary, as the BMI can be automatically calculated from weight and height values, as defined above.

Based on the BMI value, the system determines the status of the patient at step S12. The system characterizes the patient as Normal, Overweight, Obese, or Extremely Obese.

The determination is based on typical classifications where, for example, Normal is defined by a BMI of 19-24, Overweight is defined by a BMI of 25-29, Obese is defined by a BMI of 30-39, and extremely obese is defined by a BMI of 40-54.

The system allows the user to return to the data screen whereupon the system is capable of entering a CPAP screen at step S13. As with the height screen, the user could have also entered the CPAP screen initially from the data screen following step S4. The CPAP screen, through automated or manual input, allows the system to receive the CPAP titration data at step S14.

Upon receiving the weight, the height, the BMI and the CPAP titration data, the system is capable, at step S15, of graphing the information as illustrated in FIG. 1. As indicated, the data that is graphed is capable of representing the sessions accumulated over a selected time period, such as a calendar month. Alternatively, the system can graph information based on data entirely stored in memory from past sessions, and the system can graph information based upon trends from previous data to predict future behavior.

It should be noted that the present invention allows for the recording and collection of data relevant to the management of a patient participating in a BMI modification program as well as an SDB management program. The present invention provides a convenient method of presenting the patient and his clinician with both the patient's BMI measurement and his nasal CPAP therapeutic pressure changes.

The present invention has the advantage of encouraging the patient to actively participate in the management of his health conditions and thereby promote better compliance with associated treatment.

Alternatively, the invention may be used as a monitoring tool in the context of a program in which the patient undergoes SDB management by a method other than nasal CPAP. In this alternative context, the patient would undergo an infrequent nasal CPAP treatment session so as to obtain the data relevant to determining the progress of his SDB condition. This information would then be used to determine the effectiveness of the method other than nasal CPAP being used to manage his SDB.

As another alternative, the present invention may be practiced where the patient does not receive nasal CPAP or NIPPV treatment but rather his breathing (especially his breathing during sleep) is monitored but not treated. For example, the invention may be practiced in the context of a system for measuring a patient's episodes of breathing flow limitation and the calculation of the patient's AHI or AI as a function of his BMI. An example of a system that is capable of gathering and processing breathing data is the EMBLETTA Portable Diagnostic System from Flaga hg/Medcare Inc.

Various data and BMI viewing modes may be programmed to suit the particular requirements of the clinical pathway adopted for the patient. For example, in one mode the patient may be given access only to the height data and the weight data at the time it is entered while the clinician may have full access to all data entered. As a variant, the patient may have access to the BMI for a particular day but no historic data. Alternatively, the data and BMI viewing modes may be accessed only by the patient if the full data set may be accessed only by use of a secret password. The data may be displayed in any suitable form so as to suit the particular requirements of the clinical pathway adopted for the patient.

There are other ways that the data menu structure may be configured so as to address particular clinical pathway requirements. There may be included a message system that would provide encouragement, suggestions for improvement or warnings depending on how the patient's condition is progressing.

It should be emphasized that the above described embodiments of the present invention are merely specific examples. Various modifications may be made by those skilled in the art which will embody the principles of the invention and fall within the spirit and the scope thereof.

We claim:

1. A method for monitoring SDB management on a computer, the method comprising the steps of:
storing data on the computer, the data being associated with a time period of interest and relating to BMI and one or more of AHI, AI, Usage and CPAP titration; and
displaying the stored data in graphical form for a selected time period.

2. The method of claim 1 wherein the selected time period is adjustable.

3. A method for monitoring SDB management on a computer, the method comprising the steps of:
storing data on the computer, the data being associated with a time period of interest and relating to body mass and one or more of AHI, AI, Usage and CPAP titration; and
displaying the stored data in graphical form for a selected time period.

4. The method of claim 3 wherein the selected time period is adjustable.
